SEQUENCE LISTING (13 sequences)

SEQ ID NO: 1 - protein, 160 aa, Homo sapiens
MAKLTILVALALFLLAAHASARQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPQRCDLDVESGGRDRY

SEQ ID NO: 2 - protein, 172 aa, Homo sapiens
MAKLTILVALALFLLAAHASARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 3 - DNA, 40 bp, synthetic construct
  source 1..40: mol_type = other DNA
cgtggcgggggggggggcagaagggggggggggtggatta

SEQ ID NO: 4 - DNA, 40 bp, synthetic construct
  source 1..40: mol_type = other DNA
catggcgggggggggggcagaagggggggggggtggttta

SEQ ID NO: 5 - DNA, 40 bp, synthetic construct
  source 1..40: mol_type = other DNA
tgtagcgggggggggggcagaagggggggggggtggatta

SEQ ID NO: 6 - DNA, 40 bp, synthetic construct
  source 1..40: mol_type = other DNA
ggggtggcttcactttttgggtgggttgttcttttgggga

SEQ ID NO: 7 - DNA, 40 bp, synthetic construct
  source 1..40: mol_type = other DNA
tttgccagggttgaggcgtcggtggcggagatagttagag

SEQ ID NO: 8 - DNA, 40 bp, synthetic construct
  source 1..40: mol_type = other DNA
tttgggagggttgagatgggggaggcggaggtagttggag

SEQ ID NO: 9 - DNA, 40 bp, synthetic construct
  source 1..40: mol_type = other DNA
acgtaagggaccgagtgcctttggtcccttggatcgagat

SEQ ID NO: 10 - DNA, 40 bp, synthetic construct
  source 1..40: mol_type = other DNA
ggtcgggcgggtctgggcacggggtacagatgatttgcac

SEQ ID NO: 11 - DNA, 40 bp, synthetic construct
  source 1..40: mol_type = other DNA
cggtaattggacggttggctggtccgtgaacgaacccgat

SEQ ID NO: 12 - DNA, 40 bp, synthetic construct
  source 1..40: mol_type = other DNA
aaggttggcccggaggcataccttgtggtagaagacgatt

SEQ ID NO: 13 - DNA, 85 bp, synthetic construct
  source 1..85: mol_type = other DNA
tagggaagagaaggacaatgattttgggcgggttgaggtgggggaggaggaggtagttagagttgactagtacatgaccacttga

(19) United States
(12) Patent Application Publication
(10) Pub. No.: US 2026/0258428 A1
(43) Pub. Date: Sep. 3, 2026

(54) FOOD ALLERGEN PROCESSING AND DESENSITIZATION BY APTAMERS

(71) Applicant: AYASS BIOSCIENCE LLC, Frisco, TX (US)

(72) Inventors: Mohammad Ammar AYASS, San Angelo, TX (US); Lina Abi MOSLEH, Frisco, TX (US); Trivendra TRIPATHI, Frisco, TX (US); Natalya GRIKO, Frisco, TX (US); Victor PASHKOV, Frisco, TX (US); Kevin ZHU, Frisco, TX (US)

(21) Appl. No.: 18/873,038

(22) PCT Filed: Jun. 12, 2023
(86) PCT No.: PCT/US2023/068317

§ 371 Date: Dec. 9, 2024

Related U.S. Application Data
(60) Provisional application No. 63/350,932, filed on Jun. 10, 2022.

(51) Int. Cl. C12N 15/115; A61K 39/39

(52) U.S. Cl. CPC C12N 15/115; A61K 39/39; C12N 2310/16; C12N 2320/30

(57) ABSTRACT

Disclosed are aptamers and methods of their use for the treatment. Further wherein An isolated nucleic acid comprising the sequence as set forth in a specified sequence is disclosed. Wherein the nucleic acid comprises an aptamer specific for the peanut allergen Ara-H2.01 or Ara-H2.02, and wherein the RNA equivalent of the specified nucleic acid sequence is disclosed.

### I. CROSS REFERENCE TO RELATED APPLICATIONS

This application claims the benefit of U.S. Provisional Application No. 63/350,932, filed on Jun. 10, 2022, which is incorporated herein by reference in its entirety.

### II. REFERENCE TO SEQUENCE LISTING

The sequence listing submitted on Jun. 12, 2023, conforming to the rules of WIPO Standard ST.26 as an .XML file entitled “11050-008WO1.XML” created on Jun. 12, 2023, and having a file size of 4538 bytes is hereby incorporated by reference pursuant to 37 C.F.R. § 1.52(e)(5).

### III. BACKGROUND

An allergy is a condition in which the immune system reacts abnormally to a foreign substance such as food. Symptoms of a reaction can include digestive problems, hives, or swollen airways and severe reactions can be life threatening. Allergic reactions lead to approximately 200,000 emergency department visits and 200 deaths each year. What are needed are novel treatments for food allergies.

### IV. SUMMARY

Disclosed are aptamers and methods of their use for the treatment of food allergies.

In one aspect, disclosed herein are isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13.

Also disclosed herein are nucleic acids of any preceding aspect, further comprising a detectable tag (such as, for example, a latex bead, magnetic bead, fluorescence label; fluorescent probe, chemiluminescent labels, radiolabels, and/or nanoparticle probe).

In one aspect, disclosed herein are compositions comprising 1) one or more of the isolated nucleic acids of any preceding aspect and 2) water or a pharmaceutically acceptable excipient.

Also disclosed herein are kits comprising one or more of the nucleic acids of any preceding aspect.

Disclosed herein are methods of treating, inhibiting, decreasing, reducing, and/or ameliorating an ongoing allergic reaction to a food allergy (such as, for example, a peanut allergy including, but not limited to Ara-H2.01 or Ara-H2.02) in a subject comprising administering to the subject a therapeutically effective amount of the nucleic acid of any preceding aspect or the composition of any preceding aspect. For example, disclosed herein are methods of treating, inhibiting, decreasing, reducing, and/or ameliorating an ongoing allergic reaction to a food allergy (such as, for example, a peanut allergy including, but not limited to Ara-H2.01 or Ara-H2.02) in a subject comprising administering to the subject a therapeutically effective amount of one or more isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13; or a compositions comprising 1) said one or more of the isolated nucleic acids and 2) water or a pharmaceutically acceptable excipient.

Also disclosed herein are methods of immunizing a subject against a food allergy (such as, for example, a peanut allergy including, but not limited to Ara-H2.01 or Ara-H2.02) or inhibiting and/or preventing the occurrence of allergic response to a food allergen (such as, for example, a peanut allergen including, but not limited to Ara-H2.01 or Ara-H2.02) the method comprising administering to the subject a therapeutically effective amount of the nucleic acid of any of any preceding aspect or the composition of any preceding aspect. For example, disclosed herein are methods of immunizing a subject against a food allergy or inhibiting and/or preventing the occurrence of allergic response to a food allergen comprising administering to the subject a therapeutically effective amount of one or more isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13; or a compositions comprising 1) said one or more of the isolated nucleic acids and 2) water or a pharmaceutically acceptable excipient.

In one aspect, disclosed herein are methods of desensitizing a subject to a food allergen (such as, for example, a peanut allergen including, but not limited to Ara-H2.01 or Ara-H 2.02) comprising administering to the subject a therapeutically effective amount of the nucleic acid of any preceding aspect or the composition of any preceding aspect. For example, disclosed herein are methods of desensitizing a subject to a food allergen comprising administering to the subject a therapeutically effective amount of one or more isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13; or a compositions comprising 1) said one or more of the isolated nucleic acids and 2) water or a pharmaceutically acceptable excipient.

Also disclosed herein are methods of reducing, inhibiting, and/or decreasing peanut allergen (including, but not limited to Ara-H2.01 or Ara-H2.02)-induced degranulation in a subject comprising administering to the subject a therapeutically effective amount of the nucleic acid of any preceding aspect or the composition of any preceding aspect. For example, disclosed herein are methods of reducing, inhibiting, and/or decreasing peanut allergen (including, but not limited to Ara-H2.01 or Ara-H2.02)-induced degranulation in a subject comprising administering to the subject a therapeutically effective amount of one or more isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13; or a compositions comprising 1) said one or more of the isolated nucleic acids and 2) water or a pharmaceutically acceptable excipient. In one aspect, the peanut degranulation comprises ARA-H2-IgE mediated degranulation.

In one aspect, disclosed herein are methods of reducing, inhibiting, and/or decreasing the ability of a plant-based food allergen (including, but not limited to Ara-H2.01 or Ara-H2.02) to cause allergen-induced degranulation in a subject, the method comprising contacting the plant with an effective amount of the compositions of any preceding aspect or water comprising the nucleic acid of any preceding aspect. For example, disclosed herein are methods of reducing, inhibiting, and/or decreasing the ability of a plant based food allergen (including, but not limited to Ara-H2.01 or Ara-H2.02) to cause degranulation in a subject, the method comprising administering to the subject an effective amount of water or a composition, the water or composition each comprising one or more isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13.

Also disclosed herein are methods of reducing, inhibiting, and/or decreasing the ability of a plant-based food allergen (such as, for example, a peanut allergen) to cause an allergic reaction to the food comprising said allergen, the method comprising applying to the food a composition comprising any of the nucleic acids of any preceding aspect. For example, disclosed herein are methods of reducing, inhibiting, and/or decreasing the ability of a plant-based food allergen (such as, for example, a peanut allergen) to cause an allergic reaction to the food comprising said allergen, the method comprising applying to the food a composition comprising one ore more nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

### V. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1. Characterization of aptamers' binding to Ara-H2 peanut protein. ELISA based binding assay of the Ara-H2 specific aptamers (R2-11, R3-21 and R5-24) and non-specific aptamers (SB11, SB3L, Apt19, Apt20) to Ara-H2 protein. Ara-H2 peanut protein was immobilized on MaxiSorp plate and 10 nM of biotinylated aptamers were incubated with the immobilized protein. Bound aptamers were detected using Streptavidin-HRP. Each value was the average of duplicate measurements.

FIG. 2. Ara-H2 Specific aptamers inhibit degranulation in RBL-2H3 cell-line. RBL-2H3 cells (0.5×10⁶) in 100 uL EMEM media without FBS were plated in 96-well plate and sensitized with 1 ug/ml of IgE^{dansyl} (clone 27-74) for 1 hr. Natural Ara-H2 (1000 pMol) with or without Ara-H2 specific aptamers (5 uM) and unspecific aptamers (5 uM) in 100 uL cell culture media was incubated at 37° C. for 30 minutes. RBL-2H3 cells after 1 hr sensitization washed two times with EMEM media without FBS and were stimulated with the Ara-H2 with or without Ara-H2 specific or unspecific aptamers for 1 hr in 200 uL cell culture media. Degranulation was detected spectroscopically by measuring the activity of the granule-stored enzyme β-hexosaminidase secreted into the supernatant on the substrate p-nitrophenyl-N-acetyl-β-d-glucosamine. All of the degranulation assays were repeated in at least triplicate. Error bars represent mean±SD. The p-values were determined with unpaired Student t test.

FIG. 3. Crude Peanut extract induces IgE-mediated degranulation from RBL-2H3 cell-line. As shown in schematic Figure (A), we extracted proteins mixture from crude peanut and detected if crude peanut protein mixture induces IgE-mediated degranulation. Briefly, RBL-2H3 cells (0.5×10⁶) in 100 uL EMEM media without FBS were plated in 96-well plate and with or without 1 ug/ml of IgE^{dansyl} (clone 27-74) sensitized at 37° C. for 1 hr followed by washing two times with EMEM media without FBS. Cells were then stimulated with or without peanut protein at indicated serial dilutions at 37° C. for 1 hr in 200 uL cell culture media. Degranulation was detected spectroscopically by measuring the activity of the granule-stored enzyme β-hexosaminidase secreted into the supernatant on the substrate p-nitrophenyl-N-acetyl-β-d-glucosamine. All of the degranulation assays were repeated in at least triplicate. Error bars represent mean±SD. The p-values were determined with unpaired Student t test.

FIG. 4. Ara-H2 specific aptamers inhibit the degranulation induced by peanut crude proteins (PC) from RBL-2H3 cell-line. RBL-2H3 cells (0.5×10⁶) in 100 uL EMEM media without FBS were plated in 96-well plate and with or without 1 ug/ml of IgE^{dansyl} (clone 27-74) sensitized at 37° C. for 1 hr. Extracted peanut crude proteins (PC) mixture from crude peanut at dilution 1:32 with cell culture media in presence or absence of Ara-H2 specific aptamers (5 uM) and unspecific aptamers (5 uM) with aptamer and cells control conditions (as indicated) in 100 uL cell culture media was incubated at 37° C. for 30 minutes. RBL-2H3 cells after 1 hr sensitization incubation washed two times with EMEM media without FBS and were stimulated as indicated aptamers incubation conditions at 37° C. for 1 hr in 200 uL cell culture media. Degranulation was detected spectroscopically by measuring the activity of the granule-stored enzyme β-hexosaminidase secreted into the supernatant on the substrate p-nitrophenyl-N-acetyl-β-d-glucosamine. All of the degranulation assays were repeated in at least triplicate. Error bars represent mean±SD. The p-values were determined with unpaired Student t test.

FIG. 5. Ara-H2 specific aptamers inhibit the degranulation induced by Peanut crude proteins (PC) even after 64 hrs incubation on different conditions from RBL-2H3 cell-line. As shown in schematic Figure A, Extracted peanut crude proteins (PC) mixture from crude peanut at dilution 1:32 with cell culture media in presence or absence of Ara-H2 specific aptamer (5 uM) and unspecific aptamers (5 uM) in 100 uL cell culture media was incubated at 37° C. or 4° C. for 64 hr, then after degranulation assay is assessed. Briefly RBL-2H3 cells (0.5×10⁶) in 100 uL EMEM media without FBS were plated in 96-well plate and with or without 1 ug/ml of IgE^{dansyl} (clone 27-74) sensitized for 1 hr followed by washing two times with EMEM media without FBS and were stimulated as indicated aptamers incubation conditions mentioned in figure (A) at 37° C. for 1 hr in 200 uL cell culture media. Degranulation was detected spectroscopically by measuring the activity of the granule-stored enzyme β-hexosaminidase secreted into the supernatant on the substrate p-nitrophenyl-N-acetyl-β-d-glucosamine. All of the degranulation assays were repeated in at least triplicate. Error bars represent mean±SD. The p-values were determined with unpaired Student t test.

FIG. 6. Ara-H2 specific aptamers inhibit the degranulation induced by Peanut crude (PC) proteins even after 7 days incubation on room temperature from RBL-2H3 cell-line. As shown in schematic Figure A, Extracted peanut crude (PC) proteins mixture from crude peanut at dilution 1:32 with cell culture media in presence or absence of Ara-H2 specific aptamer (5 uM) and unspecific aptamers (5 uM) in 100 uL was incubated at 37° C. for 7 days, then after degranulation assay is assessed. Briefly RBL-2H3 cells (0.5×10⁶) in 100 uL EMEM media without FBS were plated in 96-well plate and with or without 1 ug/ml of IgE^{dansyl} (clone 27-74) sensitized for 1 hr followed by washing two times with EMEM media without FBS. Cells were stimulated with the aptamers incubation condition mentioned in figure (A) at 37° C. for 1 hr in 200 uL cell culture media. Degranulation was detected spectroscopically by measuring the activity of the granule-stored enzyme β-hexosaminidase secreted into the supernatant on the substrate p-nitrophenyl-N-acetyl-β-d-glucosamine. All of the degranulation assays were repeated in at least triplicate. Error bars represent mean±SD. The p-values were determined with unpaired Student t test.

FIG. 7. Peanut crude (PC) proteins extracted from peanut plants watered with Ara-H2-specific aptamers inhibit the degranulation from RBL-2H3 Cell-line. Peanut plants were treated with and without Ara-H2 specific aptamer (R3-21, R2-11, and R3-24) and control aptamers (ADI1701 and ADI 1705) 5 uM once a day for 6 months continuously. PC proteins were extracted from the peanut seeds and the degranulation assay is assessed. Briefly, RBL-2H3 cells (0.5×10⁶) in 100 uL EMEM media without FBS were plated in a 96-well plate and with or without 1 ug/ml of IgE^{dansyl} (clone 27-74) sensitized for 1 hr followed by washing two times with EMEM media without FBS. Cells were stimulated PC proteins extracted from peanut plants watered with or without Ara-H2 specific aptamers at 37° C. for 1 hr in 200 uL cell culture media. Degranulation was detected spectroscopically by measuring the activity of the granule-stored enzyme β-hexosaminidase secreted into the supernatant on the substrate p-nitrophenyl-N-acetyl-β-d-glucosamine. All of the degranulation assays were repeated in at least triplicate. Error bars represent mean±SD. The p-values were determined with an unpaired Student t-test.

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular aptamer (including, but not limited to an Ara-H2 specific aptamer) is disclosed and discussed and a number of modifications that can be made to a number of molecules including the aptamer (including, but not limited to an Ara-H2 specific aptamer) are discussed, specifically contemplated is each and every combination and permutation of aptamer (including, but not limited to an Ara-H2 specific aptamer) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

An allergy is a condition in which the immune system reacts abnormally to a foreign substance such as food. Symptoms of a reaction can include digestive problems, hives, or swollen airways and severe reactions can be life threatening. Allergic reactions lead to approximately 200,000 emergency department visits and 200 deaths each year.

Peanut allergy has been one of the fastest growing and deadliest allergies in recent history. It is responsible for more deaths from anaphylaxis than any other food allergy, with rates in children that have more than tripled in the United States in the recent years. Peanut allergies are present in the U.S. at higher rates as compared to other developed nations with more than 20% of adult Americans having reported peanut allergy.

Exposure to peanuts can occur in various ways:
1) direct contact. The most common cause of peanut allergy is eating peanuts or peanut-containing foods. Sometimes direct skin contact with peanuts can trigger an allergic reaction;
2) cross-contact. This is the unintended introduction of peanuts into a product. It's generally the result of a food being exposed to peanuts during processing or handling; and
3) Inhalation. An allergic reaction may occur if you inhale dust or aerosols containing peanuts, from a source such as peanut flour or peanut oil cooking spray.

As of 2022, there is no cure for peanut allergy other than strict avoidance of peanuts and peanut-containing foods. People with peanut allergy experience psychological, social, and economic burdens. Caregivers of children with food allergies often experience diminished quality of life, anxiety, and frustration over lack of food allergy awareness. Antihistamine drugs treat mild reactions. A severe reaction needs an injection of the drug epinephrine and emergency room care.

Aptamers known as chemical antibodies, are single-stranded DNA or RNA oligonucleotides. Because of their high specificity, sensitivity, binding affinity, and feasible alteration to the target molecule, aptamers have been deployed in many fields in medicine and agriculture. Aptamers can be a great technology for the food and agriculture industry where they can make agriculture more sustainable.

In health research, aptamers have already been used to help guide a drug to a diseased tissue while avoiding healthy cells. This facilitated the idea of using aptamers to deliver an agrochemical to a pest while avoiding any effect on the crop or other species. Aptamers can be developed to target certain food allergens and due to the high specificity to their target molecule, they can bind with high affinity and specificity to the allergen, mask the allergy stimulating motif and prevent a severe allergic reaction.

As shown herein, aptamers can be used to process food products and conceal the allergens or can alternatively be used in agriculture to water the plants with aptamers. In the latter case aptamers are taken up by the root hairs and would bind to their target allergen molecule, whether it may be a seed or the fruit.

In one aspect, disclosed herein are isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13.

Also disclosed herein are nucleic acids, further comprising a detectable tag (such as, for example, a latex bead, magnetic bead, fluorescence label; fluorescent probe, chemiluminescent labels, radiolabels, and/or nanoparticle probe).

In one aspect, disclosed herein are compositions comprising 1) one or more of the isolated nucleic acids disclosed herein and 2) water or a pharmaceutically acceptable excipient. In one aspect, the composition can be a liquid or dry (such as a powder) food additive to apply to the allergen comprising food. For example, a food additive to apply to peanuts.

Also disclosed herein are kits comprising one or more of the nucleic acids disclosed herein.

### 1. Nucleic Acids

There are a variety of molecules disclosed herein that are nucleic acid based, including for example the AraH2 aptamers disclosed herein (such as, for example, R3-21 (SEQ ID NO: 3), R2-11 (SEQ ID NO: 4), R5-24 (SEQ ID NO: 5), R2-20 (SEQ ID NO: 6), R3-6 (SEQ ID NO: 7), R4-15 (SEQ ID NO: 8), R4-49 (SEQ ID NO: 9), R7-49 (SEQ ID NO: 10), R8-6 (SEQ ID NO: 11), R10-9 (SEQ ID NO: 12), and/or SB3L (SEQ ID NO: 13)) or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### b) Primers and Probes

Disclosed are compositions including primers and probes, which are capable of interacting with the disclosed nucleic acids, such as the Ara-H2 specific aptamers disclosed herein, including, but not limited to R3-21 (SEQ ID NO: 3), R2-11 (SEQ ID NO: 4), R5-24 (SEQ ID NO: 5), R2-20 (SEQ ID NO: 6), R3-6 (SEQ ID NO: 7), R4-15 (SEQ ID NO: 8), R4-49 (SEQ ID NO: 9), R7-49 (SEQ ID NO: 10), R8-6 (SEQ ID NO: 11), R10-9 (SEQ ID NO: 12), and/or SB3L (SEQ ID NO: 13). In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### c) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively, the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a ka with the target molecule at least 10, 100, 1000, 10,000, or 100,000 fold lower than the ka with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698. Accordingly, in one aspect, disclosed herein are isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13.

Also disclosed herein are nucleic acids, further comprising a detectable tag (such as, for example, a latex bead, magnetic bead, fluorescence label; fluorescent probe, chemiluminescent labels, radiolabels, and/or nanoparticle probe).

In one aspect, disclosed herein are compositions comprising 1) one or more of the isolated nucleic acids disclosed herein and 2) water or a pharmaceutically acceptable excipient. In one aspect, the composition can comprise a liquid or dry (such as a powder) food additive to apply directly to food comprising the food allergen.

Also disclosed herein are kits comprising one or more of the nucleic acids disclosed herein.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

### 2. Peptides

### a) Protein Variants

As discussed herein there are numerous variants of the aptamers (i.e. SEQ ID Nos: 3-13) disclosed herein that are known and herein contemplated. Peptide variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is understood that for this mutation all of the nucleic acid sequences that encode this particular derivative of any of SEQ ID NOS: 3-13 are also disclosed including any degenerate nucleic acid sequences that encodes the particular polypeptide.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 3. Homology/Identity

It is understood that one way to define any known variants and derivatives or those that might arise, of the disclosed genes and proteins herein is through defining the variants and derivatives in terms of homology to specific known sequences. For example SEQ ID NOs: 3-13 set forth a particular sequence of aptamers. Specifically disclosed are variants of these aptamers herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 4. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 5. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, PA 1995.

### 6. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

The viral vectors can include nucleic acid sequence encoding a marker product. This marker product is used to determine if the gene has been delivered to the cell and once delivered is being expressed. Preferred marker genes are the E. coli lacZ gene, which encodes ß-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 7. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHODS OF USING THE COMPOSITIONS

It is understood that the disclosed aptamers (such as, for example, R3-21 (SEQ ID NO: 3), R2-11 (SEQ ID NO: 4), R5-24 (SEQ ID NO: 5), R2-20 (SEQ ID NO: 6), R3-6 (SEQ ID NO: 7), R4-15 (SEQ ID NO: 8), R4-49 (SEQ ID NO: 9), R7-49 (SEQ ID NO: 10), R8-6 (SEQ ID NO: 11), R10-9 (SEQ ID NO: 12), and/or SB3L (SEQ ID NO: 13)) can be used to alleviate allergic reactions to food allergens, inhibit allergic reactions to food allergens, or modify the ability of a food allergen to induce degranulation (including, but not limited to peanut allergen such as, for example, Ara-H2.01 or Ara-H2.02) and thereby cause an allergic reaction.

Disclosed herein are methods of treating, inhibiting, decreasing, reducing, and/or ameliorating an ongoing allergic reaction to a food allergy (such as, for example a peanut allergy) in a subject comprising administering to the subject a therapeutically effective amount of any of the nucleic acids disclosed herein or the composition comprising any one or more of said nucleic acids disclosed herein. For example, disclosed herein are methods of treating, inhibiting, decreasing, reducing, and/or ameliorating an ongoing allergic reaction to a food allergy (such as, for example a peanut allergy) in a subject comprising administering to the subject a therapeutically effective amount of one or more isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13; or a compositions comprising 1) said one or more of the isolated nucleic acids and 2) water or a pharmaceutically acceptable excipient.

Also disclosed herein are methods of immunizing a subject against a food allergy (such as, for example a peanut allergy) or inhibiting and/or preventing the occurrence of allergic response to a food allergen (such as, for example a peanut allergen including, but not limited to Ara-H2.01 or Ara-H2.02) the method comprising administering to the subject a therapeutically effective amount of any of the nucleic acids disclosed herein or the composition comprising any one or more of said nucleic acids disclosed herein; or applying to the food, any of the nucleic acids disclosed herein or the composition comprising any one or more of said nucleic acids disclosed herein. For example, disclosed herein are methods of immunizing a subject against a food allergy (such as, for example a peanut allergy) or inhibiting and/or preventing the occurrence of allergic response to a food allergen (such as, for example a peanut allergen including, but not limited to Ara-H2.01 or Ara-H2.02) comprising administering to the subject or applying directly to the food, a therapeutically effective amount of one or more isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13; or a compositions comprising 1) said one or more of the isolated nucleic acids and 2) water or a pharmaceutically acceptable excipient.

In one aspect, disclosed herein are methods of desensitizing a subject to a food allergen (such as, for example a peanut allergen including, but not limited to Ara-H2.01 or Ara-H2.02) comprising administering to the subject a therapeutically effective amount of any of the nucleic acids disclosed herein or the composition comprising any one or more of said nucleic acids disclosed herein. For example, disclosed herein are methods of desensitizing a subject to a food allergen (such as, for example a peanut allergen including, but not limited to Ara-H2.01 or Ara-H2.02) comprising administering to the subject a therapeutically effective amount of one or more isolated nucleic acids comprising an Ara-TH2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13; or a compositions comprising 1) said one or more of the isolated nucleic acids and 2) water or a pharmaceutically acceptable excipient.

Also disclosed herein are methods of reducing, inhibiting, and/or decreasing peanut allergen (including, but not limited to Ara-H2.01 or Ara-H2.02)-induced degranulation in a subject comprising administering to the subject a therapeutically effective amount of any of the nucleic acids disclosed herein or the composition comprising any one or more of said nucleic acids disclosed herein. For example, disclosed herein are methods of reducing, inhibiting, and/or decreasing peanut allergen (including, but not limited to Ara-H2.01 or Ara-H2.02)-induced degranulation in a subject comprising administering to the subject a therapeutically effective amount of one or more isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13; or a compositions comprising 1) said one or more of the isolated nucleic acids and 2) water or a pharmaceutically acceptable excipient.

In one aspect, disclosed herein are methods of reducing, inhibiting, and/or decreasing the ability of a plant-based food allergen (including, but not limited to Ara-H2.01 or Ara-H2.02) to cause degranulation in a subject, the method comprising contacting the plant with an effective amount of the compositions comprising any one or more of said nucleic acids disclosed herein or water comprising any of the nucleic acids disclosed herein. For example, disclosed herein are methods of reducing, inhibiting, and/or decreasing the ability of a plant based food allergen (including, but not limited to Ara-H2.01 or Ara-H2.02) to cause degranulation in a subject, the method comprising administering to the subject an effective amount of water or a composition, the water or composition each comprising one or more isolated nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one aspect, the nucleic acid can comprise the RNA equivalent of any of SEQ ID Nos: 3-13.

Also disclosed herein are methods of reducing, inhibiting, and/or decreasing the ability of a plant-based food allergen (such as, for example, a peanut allergen) to cause an allergic reaction to the food comprising said allergen, the method comprising applying to the food a composition comprising any of the nucleic acids disclosed herein. For example, disclosed herein are methods of reducing, inhibiting, and/or decreasing the ability of a plant-based food allergen (such as, for example, a peanut allergen) to cause an allergic reaction to the food comprising said allergen, the method comprising applying to the food a composition comprising one ore more nucleic acids comprising an Ara-H2 specific aptamer (including, but not limited to an aptamer specific for the peanut allergen Ara-H2.01 or Ara-H2.02) including, but not limited to the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Aptamers were designed through a combination of genetic selection and docking simulations. A step by step description can be found herein.

(Step 1) The protein structure of Ara h 2 was predicted using the Rosettafold web server. The sequences used for Ara h 2.01 is:

(SEQ ID NO: 1)
MAKLTILVALALFLLAAHASARQQWELQGDRRCQSQLERANLRPCEQHL
MQKIQRDEDSYERDPYSPSQDPYSPSPYDRRGAGSSQHQERCCNELNEF
ENNQRCMCEALQQIMENQSDRLQGRQQEQQFKRELRNLPQQCGLRAPQR
CDLDVESGGRDRY;
and for Ara h 2.02 is:
(SEQ ID NO: 2)
MAKLTIL VALALFLLAAHASARQQWELQGDRRCQSQLERANLRPCEQH
LMQKIQRDEDSYGRDPYSPSQDPYSPSQDPDRRDPYSPSPYDRRGAGSS
QHQERCCNELNEFENNQRCMCEALQQIMENQSDRLQGRQQEQQFKRELR
NLPQQCGLRAPQRCDLEVESGGRDRY.

(Step 2) To generate the aptamers disclosed herein, 50 aptamer sequences, each of them with a length of 40 nucleotides, were randomly generated with a A:T:G:C ratio of 1:1:1:1. (Step 3) Next, the aptamer secondary structures for the sequences generated in the previous step were predicted using DNA fold service. The secondary structures with the lowest energies were selected for folding simulations. (Step 4) The aptamer sequences generated previously and the secondary structures predicted in the previous step were used to predict the tertiary structure of the corresponding sequence. Each pair of sequence and secondary structure was used as input for RNA composer to predict the 3D structure of the given sequence; the predicted RNA 3D structures were converted to their corresponding DNA structures by replacing U with T, and by converting the ribose to deoxyribose using our in-house code. Energy minimizations were further performed for these aptamer structures. (Step 5) Next, the predicted 3D structures of Ara h2 proteins, and the aptamer structures in the prior step were used for docking simulation. The space around the DPYSPS units in both Ara h 2.01 and Ara h 2.02 were used as epitopes for docking and the docking scores were recorded. (Step 6) The aptamers with the top 15 lowest docking scores were used to generate a new batch of aptamers by randomly choosing 2 sites and randomly mutating the chosen site to a nucleotide other than the original one, for example, if the chosen site is a T, then it will be randomly mutated to A or G or C with equal likelihood. To ensure the diversity of the new batch, 40 out of the 50 new aptamers were generated using the seeds from previous batch, while the other 10 were generated randomly as in step 2, then repeating steps 3-5. (Step 7) We then repeated step 6 eight more times, for a total 10 simulated rounds of selection; and (Step 8) the best 20 aptamers were selected for further experimental verifications in the laboratory.

### List and Sequence of Ara-H2 Specific Aptamers:

| Aptamer | |
|---|---|
| Name | Aptamer Sequence |
| R3-21 | CGTGGCGGGGGGGGGGGCAGAAGGGGGGGGGGGGGATTA |
| | (SEQ ID NO: 3) |
| R2-11 | CATGGCGGGGGGGGGGGCAGAAGGGGGGGGGGGGGTTTA |
| | (SEQ ID NO: 4) |
| R5-24 | TGTAGCGGGGGGGGGGGCAGAAGGGGGGGGGGGGGATTA |
| | (SEQ ID NO: 5) |
| R2-20 | GGGGTGGCTTCACTTTTTGGGTGGGTTGTTCTTTTGGGGA |
| | (SEQ ID NO: 6) |
| R3-6 | TTTGCCAGGGTTGAGGCGTCGGTGGCGGAGATAGTTAGAG |
| | (SEQ ID NO: 7) |
| R4-15 | TTTGGGAGGGTTGAGATGGGGGAGGCGGAGGTAGTTGGAG |
| | (SEQ ID NO: 8) |
| R4-49 | ACGTAAGGGACCGAGTGCCTTTGGTCCCTTGGATCGAGAT |
| | (SEQ ID NO: 9) |
| R7-49 | GGTCGGGCGGGTCTGGGCACGGGGTACAGATGATTTGCAC |
| | (SEQ ID NO: 10) |
| R8-6 | CGGTAATTGGACGGTTGGCTGGTCCGTGAACGAACCCGAT |
| | (SEQ ID NO: 11) |
| R10-9 | AAGGTTGGCCCGGAGGCATACCTTGTGGTAGAAGACGATT |
| | (SEQ ID NO: 12) |
| SB3L | TAGGGAAGAGAAGGACAATGATTTTGGGCGGGTTGAGGTG |
| | GGGGAGGAGGAGGTAGTTAGAGTTGACTAGTACATGACCA |
| | CTTGA |
| | (SEQ ID NO: 13) |

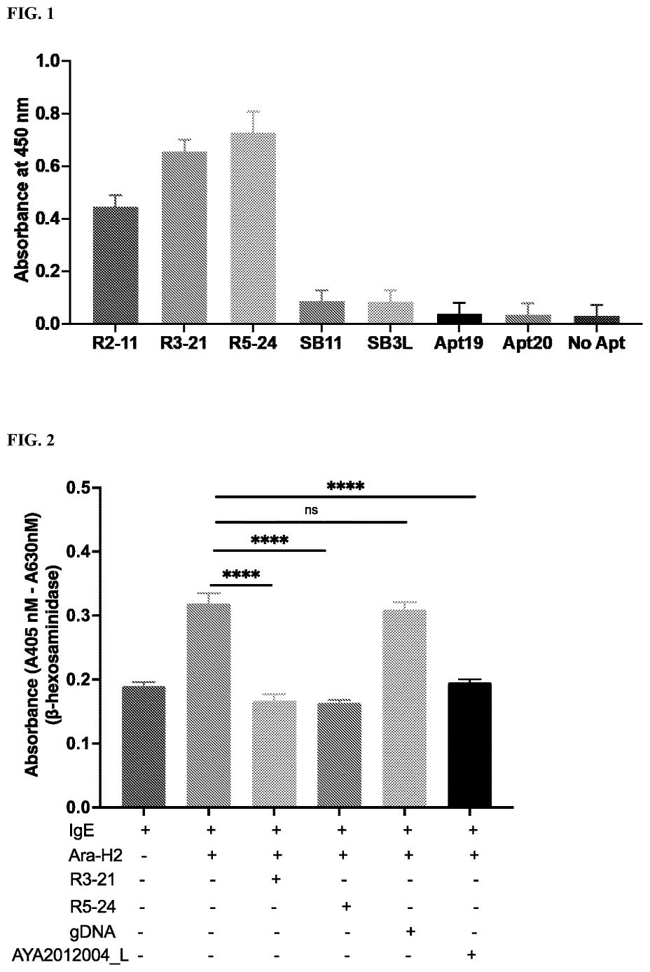

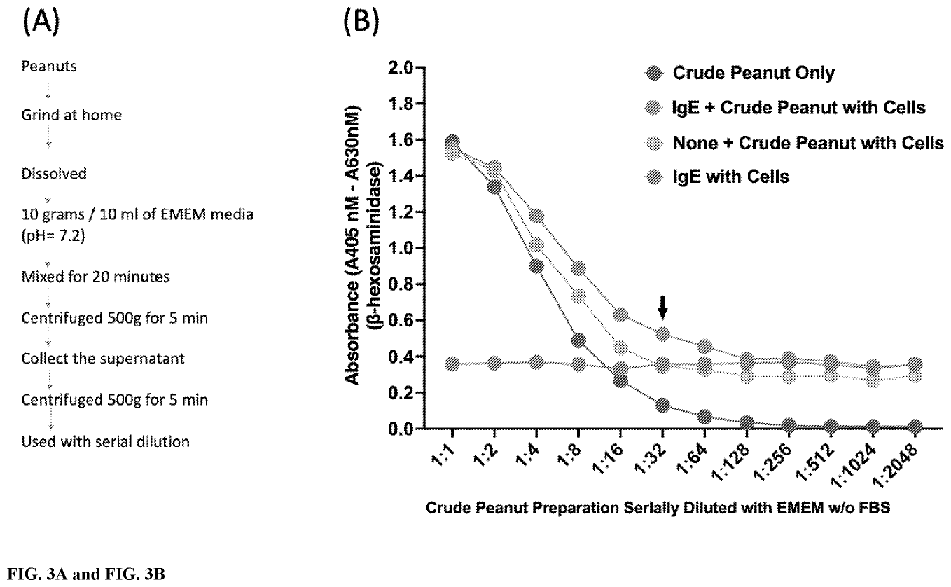

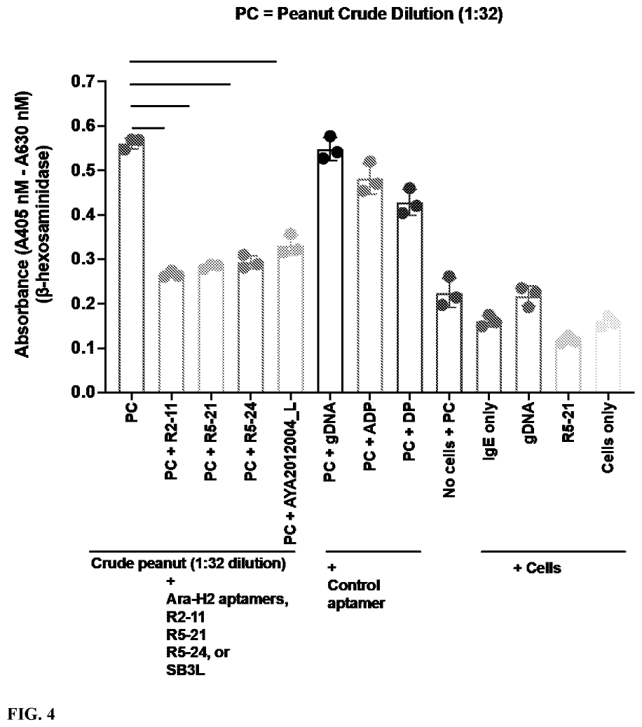

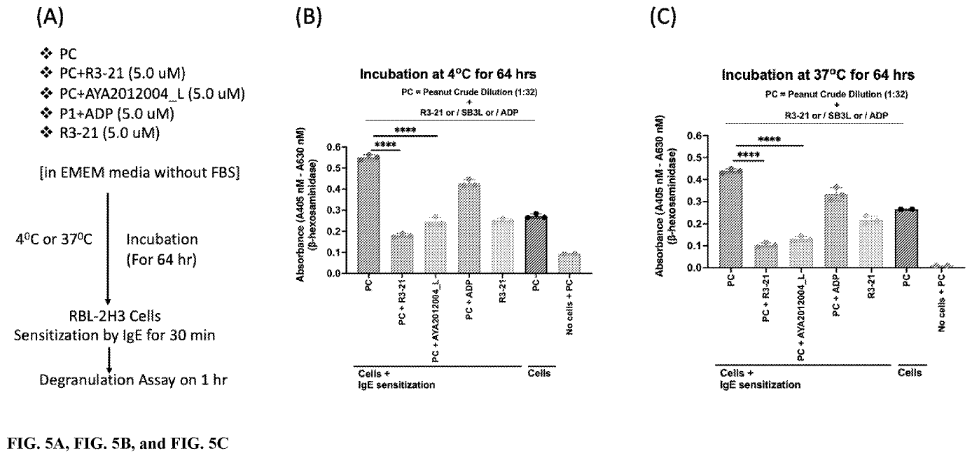

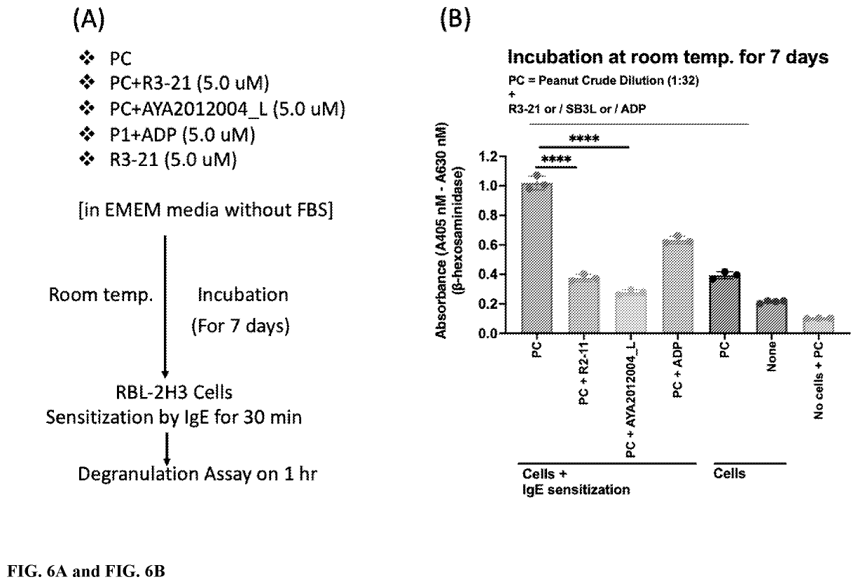

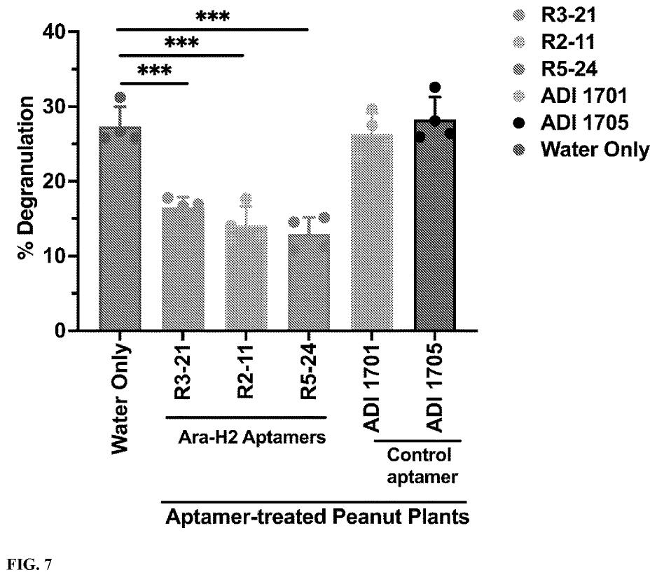

Employing the method above, we took the Ara-H2 specific aptamers and characterized their binding via ELISA (FIG. 1). Results showed that aptamer R5-24 had the most absorbance, followed by R3-21 and R2-11. Aptamers SB11, SB3L, Apt19, and Apt20 showed little binding above background levels. When then wanted to determine the ability of the Aptamers to inhibit degranulation (FIG. 2). The aptamers R5-24 and R3-21 both were able to inhibit IgE and Ara-H2 degranulation. We then measured the ability of crude peanut extract to induce IgE-mediated degranulation (FIG. 3). We were able to show that indeed, crude peanut extract can induce IgE mediated degranulation. Having established a testing model, we then looked at the ability of the aptamers to inhibit degranulation induced by crude peanut extract. We observed that the aptamers R5-24, R5-21, and R2-11 each inhibited degranulation (FIG. 4). Next we wanted to determine that ability to inhibit degranulation over time. We found that the Ara-H2 specific aptamer R3-21 was able to inhibit degranulation after 64 hours of incubation (FIG. 5) and the Ara-H2 specific aptamer was able to inhibit degranulation after 7 days of incubation (FIG. 6). Lastly, we wanted to determine if the aptamers could inhibit degranulation when applied to a plant as part of watering (FIG. 7). We observed that crude peanut extract and watered using water with R3-21, R2-11, or R5-24 was inhibited from inducing degranulation in cells stimulated with said extract. This data shows that watering with the aptamers disclosed herein can render a plant based food allergen to be hypoallergenic.

### E. REFERENCES

1. Back, M., DiMaio, F., Anishchenko, I., Dauparas, J., Ovchinnikov, S., Lee, G. R., Wang, J., Cong, Q., Kinch, L. N., Schaeffer, R. D. and Millán, C., 2021. Accurate prediction of protein structures and interactions using a three-track neural network. Science, 373(6557), pp. 871-876.
2. Mfold web server for nucleic acid folding and hybridization prediction. Nucleic Acids Res. 31 (13), 3406-15, (2003)
3. Antczak, M., Popenda, M., Zok, T., Sarzynska, J., Ratajczak, T., Tomczyk, K., Adamiak, R. W., Szachniuk, M. New functionality of RNAComposer: an application to shape the axis of miR160 precursor structure, Acta Biochimica Polonica, 2016, 63(4):737-744 (doi:10.18388/abp.2016_1329).
4. Popenda, M., Szachniuk, M., Antczak, M., Purzycka, K. J., Lukasiak, P., Bartol, N., Blazewicz, J., Adamiak, R. W. Automated 3D structure composition for large RNAs, Nucleic Acids Research, 2012, 40(14):e112

F. Sequences

SEQ ID NO: 1 Ara h 2.01 amino acid sequence
MAKLTILVALALFLLAAHASARQQWELQGDRRCQSQLERANLRPCEQH
LMQKIQRDEDSYERDPYSPSQDPYSPSPYDRRGAGSSQHQERCCNELN
EFENNQRCMCEALQQIMENQSDRLQGRQQEQQFKRELRNLPQQCGLRA
PQRCDLDVESGGRDRY;
SEQ ID NO: 1 Ara h 2.02 amino acid sequence
MAKLTIL VALALFLLAAHASARQQWELQGDRRCQSQLERANLRPCEQ
HLMQKIQRDEDSYGRDPYSPSQDPYSPSQDPDRRDPYSPSPYDRRGAG
SSQHQERCCNELNEFENNQRCMCEALQQIMENQSDRLQGRQQEQQFKR
ELRNLPQQCGLRAPQRCDLEVESGGRDRY
SEQ ID NO: 3 R3_21
CGTGGCGGGGGGGGGGGCAGAAGGGGGGGGGGGGGATTA
SEQ ID NO: 4 R2_11
CATGGCGGGGGGGGGGGCAGAAGGGGGGGGGGGGGTTTA
SEQ ID NO: 5 R5_24
TGTAGCGGGGGGGGGGGCAGAAGGGGGGGGGGGGGATTA
SEQ ID NO: 6 R2_20
GGGGGGCTTCACTTTTTGGGTGGGTTGTTCTTTTGGGGA
SEQ ID NO: 7 R3_6
TTTGCCAGGGTTGAGGCGTCGGTGGCGGAGATAGTTAGAG
SEQ ID NO: 8 R4_15
TTTGGGAGGGTTGAGATGGGGGAGGCGGAGGTAGTTGGAG
SEQ ID NO: 9 R4_49
ACGTAAGGGACCGAGTGCCTTTGGTCCCTTGGATCGAGAT
SEQ ID NO: 10 R7_49
GGTCGGGCGGGTCTGGGCACGGGGTACAGATGATTTGCAC
SEQ ID NO: 11 R8_6
CGGTAATTGGACGGTTGGCTGGTCCGTGAACGAACCCGAT
SEQ ID NO: 12 R10_9
AAGGTTGGCCCGGAGGCATACCTTGTGGTAGAAGACGATT
SEQ ID NO: 13 SB3L
TAGGGAAGAGAAGGACAATGATTTTGGGCGGGTTGAGGTGGGGGAGGA
GGAGGTAGTTAGAGTTGACTAGTACATGACCACTTGA

1. An isolated nucleic acid comprising the sequence as set forth in any of SEQ ID NO: 3, SEQ ID NO: 4, SE ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

2. The isolated nucleic acid of claim 1, wherein the nucleic acid comprises an aptamer specific for the peanut allergen Ara-H2.01 or Ara-H2.02.

3. The RNA equivalent of any of the nuclei acids of claim 1.

4. The isolated nucleic acid of claim 1, further comprising a detectable tag.

5. The isolated nucleic acid 4, wherein the detectable tag comprises a latex bead, magnetic bead, fluorescence label; fluorescent probe, chemiluminescent labels, radiolabels, and/or nanoparticle probe.

6. A composition comprising one or more of the isolated nucleic acids of claim 1 and water or a pharmaceutically acceptable excipient.

7. A kit comprising one or more of the nucleic acids of claim 1.

8. A method of treating an ongoing allergic reaction to a food allergy in a subject comprising administering to the subject a therapeutically effective amount of the nucleic acid of claim 1.

9. A method of immunizing a subject against a food allergy comprising administering to the subject a therapeutically effective amount of the nucleic acid of claim 1.

10. A method of desensitizing a subject to a food allergen comprising administering to the subject a therapeutically effective amount of the nucleic acid of claim 1.

11. A method of reducing peanut allergen-induced degranulation in a subject comprising administering to the subject a therapeutically effective amount of the nucleic acid of claim 1.

12. The method of claim 11, wherein the peanut allergen-induced degranulation comprises ARA-H2-IgE mediated degranulation.

13. A method of reducing the ability of a plant-based food allergen to cause allergen-induced degranulation in a subject, the method comprising contacting the plant with an effective amount of the compositions of claim 6 or water comprising the nucleic acid of claim 1.

14. A method of inhibiting the ability of a plant-based food allergen to cause an allergic reaction to the food comprising said allergen, the method comprising applying to the food, the composition of claim 6.

15. The method of claim 8, wherein the allergen is a peanut allergen, and the food allergy is a peanut allergy.

16. The method of claim 15, wherein the peanut allergen comprises Ara-H2.01 or Ara-H2.02.

17. The method of claim 9, wherein the allergen is a peanut allergen, and the food allergy is a peanut allergy.

18. The method of claim 10, wherein the allergen is a peanut allergen, and the food allergy is a peanut allergy.

19. The method of claim 11, wherein the allergen is a peanut allergen, and the food allergy is a peanut allergy.

20. The method of claim 13, wherein the allergen is a peanut allergen, and the food allergy is a peanut allergy.
